(19) United States
(12) Patent Application Publication
(10) Pub. No.: US 2025/0352370 A1
(43) Pub. Date: Nov. 20, 2025

(54) ENDOPROSTHESIS WITH IMPROVED CHARACTERISTICS

(71) Applicant: BOSTON SCIENTIFIC SCIMED, INC., Maple Grove, MN (US)

(72) Inventors: DANIEL TUCK, Galway (IE); GARY GILMARTIN, Foxford (IE); FIONN STAPLETON, Galway (IE); THOMAS MARTIN KEATING, Galway (IE); CELINE GLYNN, Galway (IE); JOHN THOMAS O'DRISCOLL, Galway (IE); HARRY MULLEADY, Galway (IE)

(73) Assignee: BOSTON SCIENTIFIC SCIMED, INC., Maple Grove, MN (US)

(21) Appl. No.: 19/208,676

(22) Filed: May 15, 2025

Related U.S. Application Data
(60) Provisional application No. 63/648,355, filed on May 16, 2024.

(51) Int. Cl. A61F 2/89

(52) U.S. Cl. CPC A61F 2/89; A61F 2220/0075; A61F 2230/0006; A61F 2240/001

(57) ABSTRACT

An endoprosthesis, such as a stent, includes a body portion extending axially from a first end to a second end along a central longitudinal axis; wherein the body portion is formed from a single wire extending from the first end to the second end and where the single wire forms a plurality of circumferential segments extending non-helically around the central longitudinal axis; and a reinforcing filament extending substantially longitudinally along the body portion.

### CROSS-REFERENCE TO RELATED APPLICATIONS

The application claims the benefit of U.S. Provisional Patent Application Ser. No. 63/648,355, filed on May 16, 2024, the disclosure of which is incorporated herein by reference.

### TECHNICAL FIELD

The present disclosure pertains to medical devices, methods for manufacturing medical devices, and uses thereof. More particularly, the present disclosure pertains to an endoprosthesis or stent for implantation in a body lumen, and associated methods.

### BACKGROUND

An endoprosthesis may be used in the treatment of body lumens. One type of endoprosthesis used in the repair and/or treatment of diseases in various body lumens is a stent. A stent is a generally longitudinal tubular device formed of biocompatible material which is useful to open and support various lumens in the body. For example, stents may be used in the vascular system, urogenital tract, gastrointestinal tract, esophageal tract, tracheal/bronchial tubes, and bile duct, as well as in a variety of other applications in the body.

In some instances, it may be desirable to design an endoprosthesis to include sufficient flexibility and conformability to the body lumen, while maintaining sufficient radial force to open the body lumen at the treatment site and/or prevent migration of the endoprosthesis within the body lumen. In some instances, it may be desirable to reduce or limit foreshortening. In some instances, different endoprosthesis configurations may provide different deliverability, flexibility, conformability (e.g., to a body lumen), radial force/strength, and/or anchoring/migration characteristics.

Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices.

A first example is an endoprosthesis, comprising: a body portion extending axially from a first end to a second end along a central longitudinal axis; wherein the body portion is formed from a single wire extending from the first end to the second end; and wherein the single wire forms a plurality of circumferential segments extending non-helically around the central longitudinal axis; and a reinforcing filament extending substantially longitudinally along the body portion.

Alternatively or additionally to any of the examples herein, in another example, wherein the reinforcing filament has a length that is substantially equal to a length of the body portion.

Alternatively or additionally to any of the examples herein, in another example, wherein the reinforcing filament is interwoven with the plurality of circumferential segments of the single wire.

Alternatively or additionally to any of the examples herein, in another example, wherein the reinforcing filament is interwoven in an alternating over and under fashion with the plurality of circumferential segments of the single wire.

Alternatively or additionally to any of the examples herein, in another example, wherein the reinforcing filament is in direct contact with the plurality of circumferential segments.

Alternatively or additionally to any of the examples herein, in another example, wherein the plurality of circumferential segments includes a plurality of medial circumferential segments, and wherein the reinforcing filament is interwoven with at least each of the plurality of medial circumferential segments.

Alternatively or additionally to any of the examples herein, in another example, wherein the reinforcing filament is an elongated planar strip.

Alternatively or additionally to any of the examples herein, in another example, wherein the reinforcing filament is formed of polyester, polytetrafluoroethylene, or a combination thereof.

Alternatively or additionally to any of the examples herein, in another example, wherein the reinforcing filament is included in a plurality of reinforcing filaments, and wherein each of the plurality of reinforcing filaments extends substantially longitudinally along the body portion.

Alternatively or additionally to any of the examples herein, in another example, wherein respective reinforcing filaments of the plurality of reinforcing filaments are spaced apart circumferentially about the body portion.

Alternatively or additionally to any of the examples herein, in another example, wherein the plurality of reinforcing filaments comprise: a first reinforcing filament and a second reinforcing filament, wherein the first reinforcing filament and the second reinforcing filament are spaced apart circumferentially about the body portion.

Alternatively or additionally to any of the examples herein, in another example, wherein respective reinforcing filaments of the plurality of reinforcing filaments are the same shape.

Alternatively or additionally to any of the examples herein, in another example, wherein respective reinforcing filaments of the plurality of reinforcing filaments are the same size.

Alternatively or additionally to any of the examples herein, in another example, further comprising a polymeric covering fixedly attached to the body portion, wherein the reinforcing filament is embedded in the polymeric covering.

Alternatively or additionally to any of the examples herein, in another example, wherein a length of the body portion from the first end to the second end is configured to change by less than 5% when shifting between a collapsed delivery configuration and an expanded deployed configuration.

Another example is a self-expanding endoprosthesis, comprising: a body portion extending axially from a first end to a second end along a central longitudinal axis; wherein the body portion is formed from a single wire extending from the first end to the second end; and wherein the single wire forms a plurality of circumferential segments extending non-helically around the central longitudinal axis; a plurality of reinforcing filaments extending substantially longitudinally along the body portion, wherein respective reinforcing filaments of the plurality of reinforcing filaments are interwoven with the plurality of circumferential segments and are formed of a different material than the body portion; and a polymeric covering fixedly attached to the body portion, wherein the plurality of reinforcing filaments and the body portion are embedded in the polymeric covering.

Alternatively or additionally to any of the examples herein, in another example, wherein: each circumferential segment of the plurality of circumferential segments comprises an undulating arrangement of first struts and second struts defining peaks and valleys, the first struts being disposed at a first angle relative to the central longitudinal axis in a side view of the body portion and the second struts being disposed at a second angle relative to the central longitudinal axis in the side view of the body portion; each circumferential segment of the plurality of circumferential segments is connected to an axially adjacent circumferential segment of the plurality of circumferential segments by a transition segment of the single wire disposed at a third angle relative to the central longitudinal axis in the side view of the body portion, wherein the third angle is different from the first angle and the second angle; and all transition segments of the single wire collectively define a transition zone.

Alternatively or additionally to any of the examples herein, in another example, wherein the transition zone extends: parallel to the central longitudinal axis; and helically around the central longitudinal axis.

Another examples is a method of forming an endoprosthesis, the method comprising: receiving a body portion extending axially from a first end to a second end along a central longitudinal axis; wherein the body portion is formed from a single wire extending from the first end to the second end; and wherein the single wire forms a plurality of circumferential segments extending non-helically around the central longitudinal axis; interweaving a reinforcing filament with the plurality of circumferential segments of the single wire to form a reinforced framework, wherein the reinforcing filament is formed of a different material than the body portion; and covering the reinforced framework with a polymeric covering to form the endoprosthesis, wherein a length of the body portion from the first end to the second end is configured to change by less than 5% when shifting between a collapsed delivery configuration and an expanded deployed configuration.

Alternatively or additionally to any of the examples herein, in another example, wherein covering the reinforced framework with the polymeric covering further comprises applying a liquid polymeric covering to the reinforced framework.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1A is a flat pattern view illustrating selected aspects of an endoprosthesis in accordance with the disclosure;

FIG. 1B is a view of a portion of the endoprosthesis in FIG. 1A;

FIG. 1C is a section view of the portion of the endoprosthesis in FIG. 1B;

FIG. 1D is another section view of the portion of the endoprosthesis in FIG. 1B;

FIG. 2 is a flat pattern view illustrating selected aspects of an endoprosthesis in accordance with the disclosure;

FIG. 3 is a flat pattern view illustrating selected aspects of an endoprosthesis in accordance with the disclosure;

FIG. 4 is a flat pattern view illustrating selected aspects of an endoprosthesis in accordance with the disclosure;

FIG. 5 is a flat pattern view illustrating selected aspects of an endoprosthesis in accordance with the disclosure;

FIG. 6 is a flat pattern view illustrating selected aspects of an endoprosthesis in accordance with the disclosure;

FIG. 7 illustrates selected aspects of an endoprosthesis in accordance with the disclosure using side and end views; and

FIGS. 8A-8B schematically illustrate selected aspects of an endoprosthesis system in accordance with the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

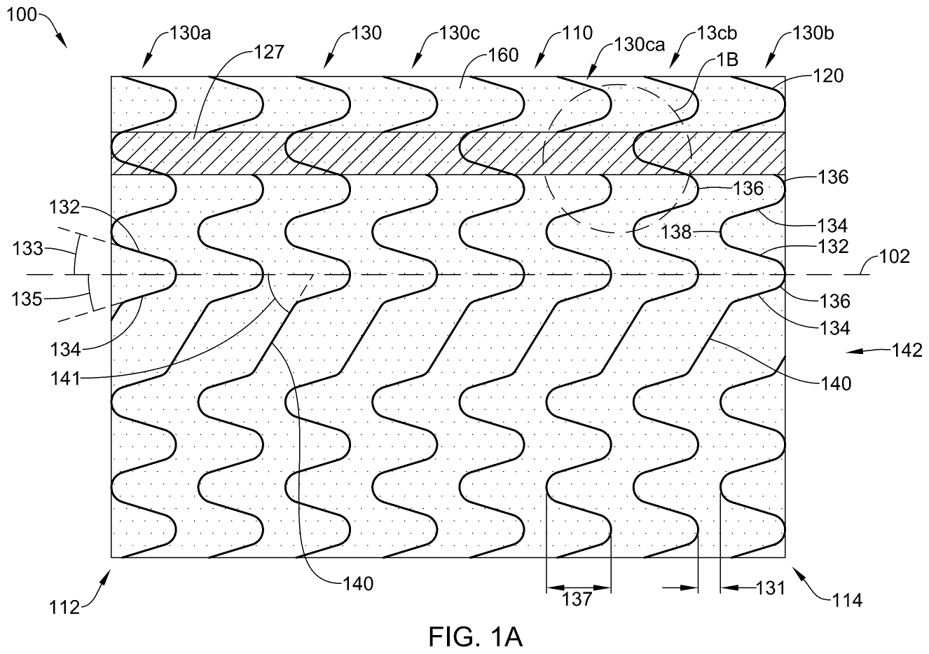

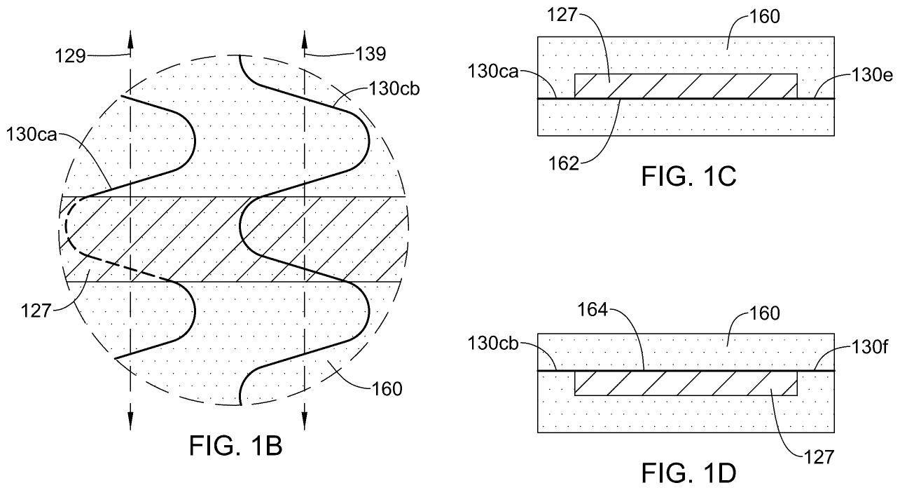

FIGS. 1A-1D and 2-7 illustrate selected aspects of an endoprosthesis 100, such as a stent, wherein FIGS. 1A-1D and 2-6 illustrate the endoprosthesis 100 in a flat pattern view. The endoprosthesis 100 may comprise a body portion 110 extending axially from a first end 112 to a second end 114 along a central longitudinal axis 102. In some embodiments, the first end 112 and the second end 114 may define a length of the endoprosthesis 100 and/or the body portion 110. In at least some embodiments, the body portion 110 may be tubular. The body portion 110 may define a lumen extending therethrough from the first end 112 to the second end 114.

In some embodiments, the endoprosthesis 100 may comprise a flared first end portion (not shown) and/or a flared second end portion (not shown), wherein an outer diameter of the flared first end portion is greater than an outer diameter of the body portion 110 and/or an outer diameter of the flared second end portion is greater than the outer diameter of the body portion 110. Other configurations are also contemplated. The flared first end portion may be disposed proximate and/or adjacent to the first end 112. In some embodiments, the flared first end portion may extend from the first end 112 to the body portion 110. The flared second end portion may be disposed proximate and/or adjacent to the second end 114. In some embodiments, the flared second end portion may extend from the second end 114 to the body portion 110.

In some embodiments, the outer diameter of the body portion 110 may be generally uniform and/or generally constant along the length of the endoprosthesis 100 and/or the body portion 110. In some embodiments, the outer diameter of the body portion 110 may be generally uniform and/or generally constant along the length of the body portion 110 except for the flared first end portion and/or the flared second end portion, where present. In some embodiments, the outer diameter of the body portion 110 may be generally uniform and/or generally constant along the length of the body portion 110 from the flared first end portion to the flared second end portion, where present. In some embodiments, an outer diameter of the body portion 110 can be in a range from 4 to 40 millimeters, among other possibilities.

In some embodiments, the endoprosthesis 100 and/or the body portion 110 may be formed from a single wire 120 extending from the first end 112 to the second end 114. In some embodiments, the flared first end portion and/or the flared second end portion, where present, may be formed from the single wire 120. The single wire 120 may extend circumferentially around the central longitudinal axis 102 and/or the single wire 120 may encircle the central longitudinal axis 102. The single wire 120 is and/or consists of only one wire.

In some embodiments, the single wire 120 may form a plurality of circumferential segments 130 extending non-helically around the central longitudinal axis 102. In some embodiments, the plurality of circumferential segments 130 may be substantially parallel to each other. In some embodiments, each circumferential segment of the plurality of circumferential segments 130 may form a ring extending around the central longitudinal axis 102. Some suitable but non-limiting examples of materials for the endoprosthesis 100 and/or the single wire 120, such as metallic materials, composite materials, shape memory materials, combinations thereof, etc., are discussed below.

In some embodiments, the plurality of circumferential segments 130 may comprise a first end circumferential segment 130a forming the first end 112, a second end circumferential segment 130b forming the second end 114, and at least one medial circumferential segment 130c disposed axially between the first end circumferential segment 130a and the second end circumferential segment 130b. In some embodiments, the at least one medial circumferential segment 130c may comprise one circumferential segment, two circumferential segments, three circumferential segments, etc. up to a desired number of circumferential segments producing a desired overall length for the endoprosthesis 100.

In some embodiments, each circumferential segment of the plurality of circumferential segments 130 may comprise an undulating arrangement (e.g., a sinusoidal arrangement) of first struts 132 and second struts 134 defining peaks 136 and valleys 138. In at least some embodiments, within any particular circumferential segment of the plurality of circumferential segments 130, the first struts 132 have a first length defined by the peaks 136 and the valleys 138 and the second struts 134 have a second length defined by the peaks 136 and the valleys 138, wherein the first length is equal to the second length. In some embodiments, the first length and the second length may be uniform and/or constant among the plurality of circumferential segments 130. In some alternative embodiments, the first length and the second length may vary among the plurality of circumferential segments 130.

In some embodiments, the first struts 132 may be disposed at a first angle 133 relative to the central longitudinal axis 102 in a side view and/or a flat pattern view of the body portion 110 and/or the endoprosthesis 100, and the second struts 134 may be disposed at a second angle 135 relative to the central longitudinal axis 102 in the side view and/or the flat pattern view of the body portion 110 and/or the endoprosthesis 100. In at least some embodiments, the second angle may be different from the first angle. The first angle and the second angle can be in a range zero to 90 degrees, among other possibilities.

In some embodiments, the peaks 136 and the valleys 138 of adjacent circumferential segments of the plurality of circumferential segments 130 may be aligned with each other axially along the length of the body portion 110 and/or the endoprosthesis 100. For example, the peaks 136 of one circumferential segment of the plurality of circumferential segments 130 may be aligned axially with the peaks 136 of an immediately adjacent circumferential segment of the plurality of circumferential segments 130. Similarly, the valleys 138 of one circumferential segment of the plurality of circumferential segments 130 may be aligned axially with the valleys 138 of an immediately adjacent circumferential segment of the plurality of circumferential segments 130. In some embodiments, the peaks 136 and the valleys 138 of adjacent circumferential segments of the plurality of circumferential segments 130 may be aligned parallel to the central longitudinal axis 102. Other configurations are also contemplated.

In some embodiments, the peaks 136 and the valleys 138 of each circumferential segment of the plurality of circumferential segments 130 may be aligned with each other axially along the length of the body portion 110 and/or the endoprosthesis 100. For example, the peaks 136 of each circumferential segment of the plurality of circumferential segments 130 may be aligned axially with the peaks 136 of each other circumferential segment of the plurality of circumferential segments 130. Similarly, the valleys 138 of each circumferential segment of the plurality of circumferential segments 130 may be aligned axially with the valleys 138 of each other circumferential segment of the plurality of circumferential segments 130. In some embodiments, the peaks 136 and the valleys 138 of each circumferential segment of the plurality of circumferential segments 130 may be aligned parallel to the central longitudinal axis 102. Other configurations are also contemplated.

In some embodiments, each adjacent pair of circumferential segments of the plurality of circumferential segments 130 defines an axial spacing 131 therebetween. The axial spacing 131 may be measured axially and/or parallel to the central longitudinal axis 102. In some embodiments, the axial spacing 131 may be defined between the valleys 138 of one circumferential segment and the peaks 136 of an immediately adjacent circumferential segment. For the purpose of this disclosure, the axial spacing 131 shall be understood to not include any axial overlap with the first struts 132 and/or the second struts 134 of any given circumferential segment.

In some embodiments, the axial spacing 131 between adjacent pairs of circumferential segments of the plurality of circumferential segments 130 may be uniform along the length of the body portion 110 and/or the endoprosthesis 100, as seen in FIG. 1A. In some embodiments, adjacent pairs of circumferential segments of the plurality of circumferential segments 130 are equally and/or uniformly spaced apart along the length of the body portion 110 and/or the endoprosthesis 100.

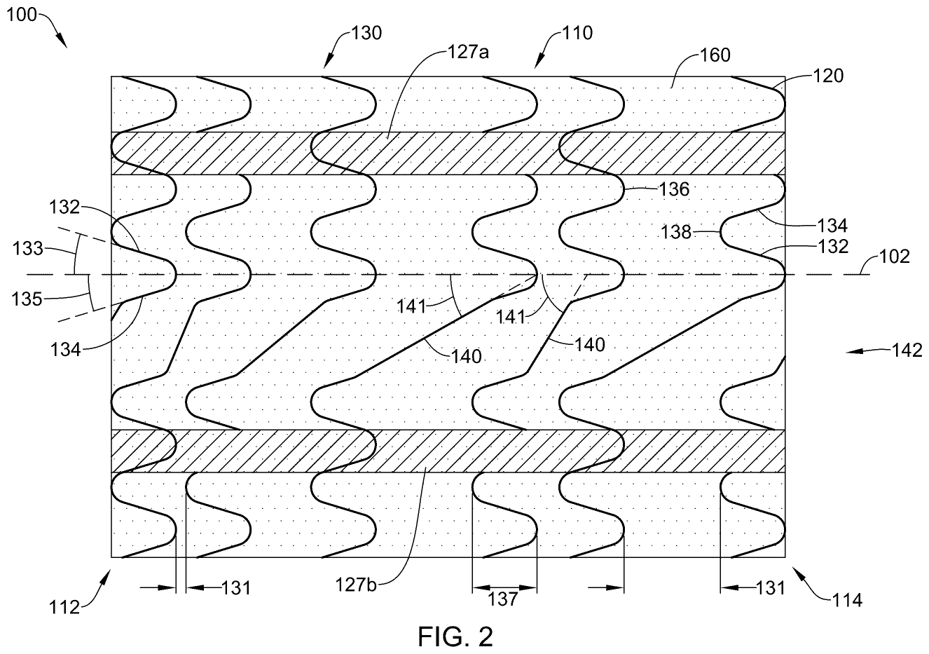

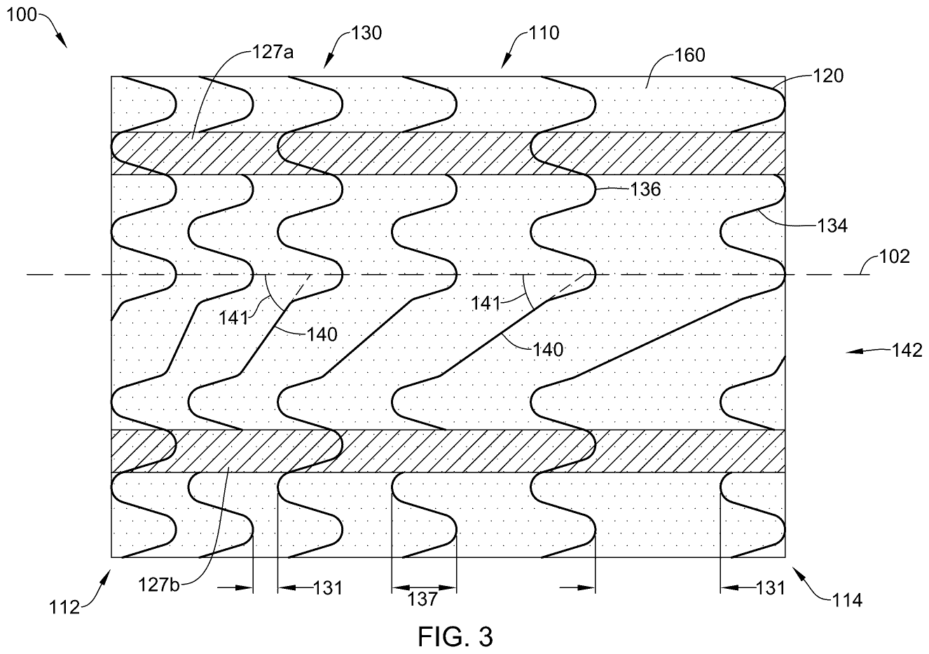

In some embodiments, the axial spacing 131 between adjacent pairs of circumferential segments of the plurality of circumferential segments 130 may vary along the length of the body portion 110 and/or the endoprosthesis 100, as seen in FIG. 2. In some embodiments, adjacent pairs of circumferential segments of the plurality of circumferential segments 130 are unequally and/or non-uniformly spaced apart along the length of the body portion 110 and/or the endoprosthesis 100. In some embodiments, the axial spacing 131 between adjacent pairs of circumferential segments of the plurality of circumferential segments 130 may increase along the length of the body portion 110 and/or the endoprosthesis 100 from the first end 112 toward and/or to the second end 114, as seen in FIG. 3. Alternatively, in some embodiments, the axial spacing 131 between adjacent pairs of circumferential segments of the plurality of circumferential segments 130 may decrease along the length of the body portion 110 and/or the endoprosthesis 100 from the first end 112 toward and/or to the second end 114.

In some embodiments, the peaks 136 and the valleys 138 of each circumferential segment of the plurality of circumferential segments 130 may define a circumferential segment height 137 for that circumferential segment measured parallel to the central longitudinal axis 102. In some embodiments, the circumferential segment height 137 may be uniform along the length of the body portion 110 and/or the endoprosthesis 100 and/or among the plurality of circumferential segments 130, as seen in FIG. 1A.

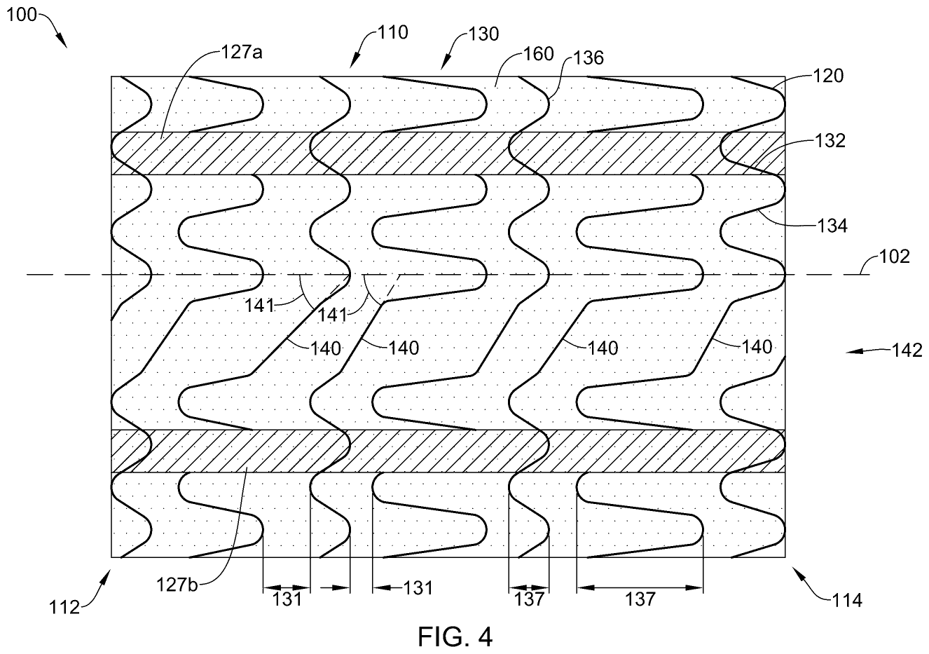

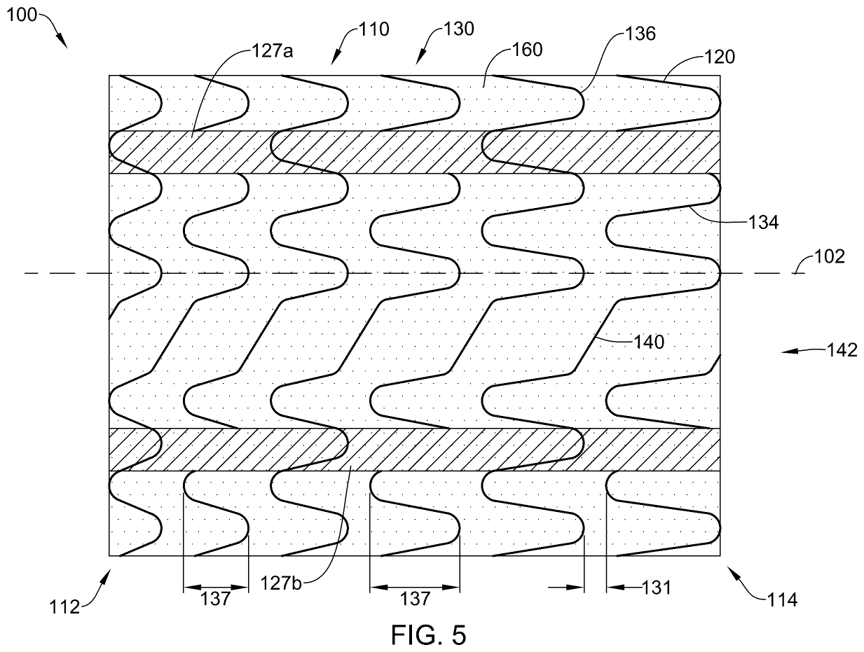

In some embodiments, the circumferential segment height 137 may vary along the length of the body portion 110 and/or the endoprosthesis 100 and/or among the plurality of circumferential segments 130, as seen in FIG. 4. In some embodiments, the circumferential segment height 137 may increase along the length of the body portion 110 and/or the endoprosthesis 100 and/or among the plurality of circumferential segments 130 from the first end 112 toward and/or to the second end 114, as seen in FIG. 5. Alternatively, in some embodiments, the circumferential segment height 137 may decrease along the length of the body portion 110 and/or the endoprosthesis 100 and/or among the plurality of circumferential segments 130 from the first end 112 toward and/or to the second end 114.

Returning to FIG. 1A, in some embodiments, each circumferential segment of the plurality of circumferential segments 130 may be connected to an axially adjacent circumferential segment of the plurality of circumferential segments 130 by a transition segment 140 of the single wire 120. In at least some embodiments, each circumferential segment of the plurality of circumferential segments 130 may be discontinuous and/or may extend around less than a full circumference of the endoprosthesis 100 and/or the body portion 110. Instead, each circumferential segment of the plurality of circumferential segments 130 may have a first end and a second end, wherein the transition segment 140 extends between a first end of one circumferential segment of the plurality of circumferential segments 130 and a second end of an immediately adjacent circumferential segment of the plurality of circumferential segments 130 (or the transition segment 140 extends between a second end of one circumferential segment of the plurality of circumferential segments 130 and a first end of an immediately adjacent circumferential segment of the plurality of circumferential segments 130). In some embodiments, at least a portion of the first end circumferential segment 130a and/or the second end circumferential segment 130b may extend completely around the full circumference of the endoprosthesis 100 and/or the body portion 110, and the at least one medial circumferential segment 130c may be discontinuous and/or may extend around less than the full circumference of the endoprosthesis 100 and/or the body portion 110.

The transition segment 140 of the single wire 120 may be disposed at a third angle 141 relative to the central longitudinal axis 102 in the side view and/or the flat pattern view of the body portion 110 and/or the endoprosthesis 100. In at least some embodiments, the third angle 141 may be different from the first angle 133 and the second angle 135. The lengths of the transition segment can be in a range from about 5 millimeters to about 20 millimeters, in some instances. In some embodiments, each circumferential segment of the plurality of circumferential segments 130 may be connected to an axially adjacent circumferential segment of the plurality of circumferential segments 130 by a transition segment 140 of the single wire 120 disposed nonparallel to the central longitudinal axis 102, the first struts 132, and the second struts 134 in the side view and/or the flat pattern view of the body portion 110 and/or the endoprosthesis 100. Other configurations are also contemplated.

In some embodiments, the transition segment 140 extending and/or disposed between axially adjacent circumferential segments of the plurality of circumferential segments 130 may extend between a valley (ref. 138) of one circumferential segment and a peak (ref. 136) of an immediately axially adjacent circumferential segment (or between a peak of one circumferential segment and a valley of an immediately axially adjacent circumferential segment). As illustrated, the transition segment(s) 140 extend from a peak (ref. 136) of a first circumferential segment disposed closer to the first end 112 to a valley (ref. 138) of a second circumferential segment immediately axially adjacent the first circumferential segment that is disposed closer to the second end 114 than the first circumferential segment. Other configurations are also contemplated. In some embodiments, the transition segment 140 may be disposed nonparallel with the first struts 132 and the second struts 134 of one circumferential segment (e.g., the first circumferential segment) and the first struts 132 and the second struts 134 of the immediately axially adjacent circumferential segment (e.g., the second circumferential segment).

In some embodiments, all transition segments (e.g., an array of transition segments consisting of each transition segment 140 connecting and/or extending between adjacent circumferential segments of the plurality of circumferential segments 130) of the single wire 120 may collectively define a transition zone 142. In some embodiments, the transition zone 142 may extend parallel to the central longitudinal axis 102, as seen in FIG. 1A. In some embodiments, the third angle 141 may be uniform along and/or within the transition zone 142.

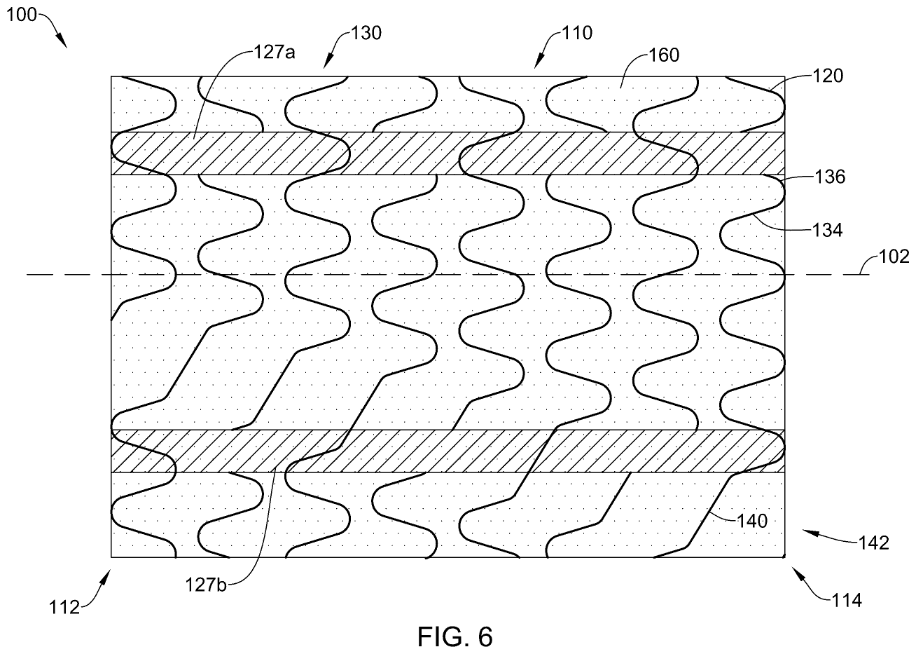

In some embodiments, each transition segment 140 of the single wire 120 may be aligned axially along the length of the body portion 110 and/or the endoprosthesis 100. In some embodiments, the transition zone 142 may define a preferential bending plane for the endoprosthesis 100 and/or the body portion 110 (e.g., the endoprosthesis 100 and/or the body portion 110 may be configured to bend more easily along and/or within the transition zone 142 than in other directions). In some embodiments, the third angle 141 may vary along and/or within the transition zone 142, as seen in FIGS. 2-3 for example. In some embodiments, the transition zone 142 may extend helically along and/or around the central longitudinal axis 102, as seen in FIG. 6.

In some embodiments, the peaks 136 and the valleys 138 of adjacent circumferential segments of the plurality of circumferential segments 130 may extend helically along the length of the body portion 110 and/or the endoprosthesis 100. In some embodiments, the peaks 136 and the valleys 138 of adjacent circumferential segments of the plurality of circumferential segments 130 may extend helically along and/or around the central longitudinal axis 102. In some embodiments, the peaks 136 and the valleys 138 of each circumferential segment of the plurality of circumferential segments 130 may extend helically along the length of the body portion 110 and/or the endoprosthesis 100. In some embodiments, the peaks 136 and the valleys 138 of each circumferential segment of the plurality of circumferential segments 130 may extend helically along and/or around the central longitudinal axis 102. Other configurations are also contemplated.

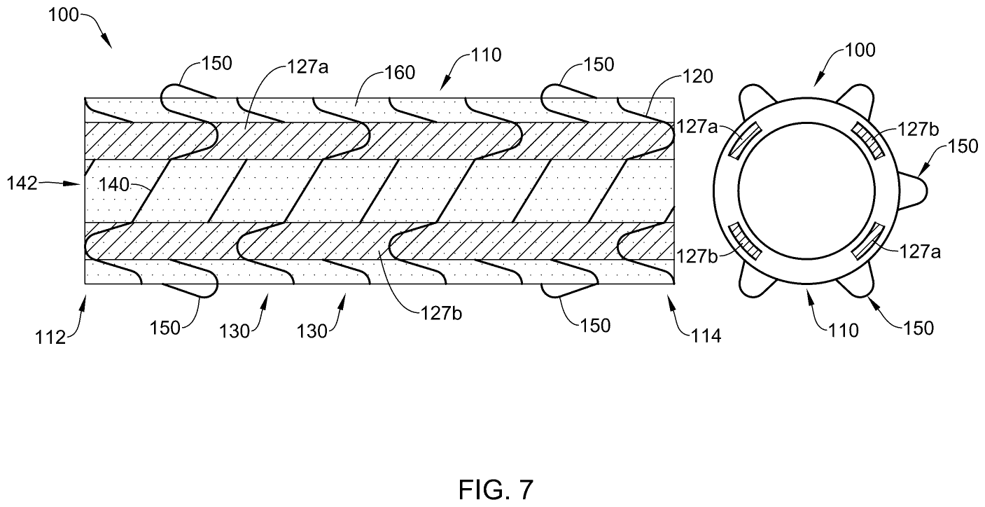

In some embodiments, the endoprosthesis 100 and/or the body portion 110 may comprise one or more anti-migration elements disposed along the length of the endoprosthesis 100 and/or the body portion 110. In some embodiments, at least one circumferential segment of the plurality of circumferential segments 130 may comprise an anti-migration element 150 projecting radially outward from the at least one circumferential segment and/or an outer surface of the endoprosthesis 100 and/or the body portion 110, as seen in FIG. 7. In some embodiments, at least one circumferential segment of the plurality of circumferential segments 130 may comprise a plurality of anti-migration elements projecting radially outward from the at least one circumferential segment and/or the outer surface of the endoprosthesis 100 and/or the body portion 110. As mentioned, the endoprosthesis 100 can include a plurality of reinforcing filaments 127, as illustrated in FIG. 7.

In some embodiments, anti-migration element(s) positioned proximate the first end 112 may project radially outward and toward the second end 114 (e.g., at a non-zero angle relative to the outer surface of the endoprosthesis 100 and/or the body portion 110) and/or anti-migration element(s) positioned proximate the second end 114 may project radially outward and toward the first end 112 (e.g., at a non-zero angle relative to the outer surface of the endoprosthesis 100 and/or the body portion 110). In some embodiments, anti-migration element(s) positioned proximate the first end 112 may project radially outward and toward the first end 112 (e.g., at a non-zero angle relative to the outer surface of the endoprosthesis 100 and/or the body portion 110) and/or anti-migration element(s) positioned proximate the second end 114 may project radially outward and toward the second end 114 (e.g., at a non-zero angle relative to the outer surface of the endoprosthesis 100 and/or the body portion 110). In some embodiments, some anti-migration elements positioned proximate the first end 112 may project toward the first end 112 and some may project the second end 114 (e.g., at a non-zero angle relative to the outer surface of the endoprosthesis 100 and/or the body portion 110), and/or some anti-migration elements positioned proximate the second end 114 may project toward the first end 112 and some may project the second end 114 (e.g., at a non-zero angle relative to the outer surface of the endoprosthesis 100 and/or the body portion 110). Other configurations are also contemplated.

The endoprosthesis 100 may comprise a reinforcing filament 127. In some embodiments, the reinforcing filament 127 may be formed of polyester, polytetrafluoroethylene (PTFE), or a combination thereof, among other suitable materials such as those detailed herein. In some embodiments, the reinforcing filament 127 may be formed of polyester. In some embodiments, the reinforcing filament 127 may be formed of PTFE.

The reinforcing filament 127 may have a uniform width taken along an entire length (e.g., extending longitudinally along the central longitudinal axis of the endoprosthesis 100), as illustrated in FIG. 1A. However, in some embodiments, the reinforcing filament 127 can have a variable width (e.g., different widths taken at two or more positions along the length of the reinforcing filament 127).

The reinforcing filament 127 may have a uniform width and/or uniform thickness. For instance, the reinforcing filament 127 may have a uniform thickness (e.g., in the direction normal to the central longitudinal axis of the endoprosthesis 100) along an entire length (e.g., in a direction along or coaxial with the central longitudinal axis of the endoprosthesis 100) of the reinforcing filament. However, in some embodiments, the reinforcing filament 127 can have a variable thickness (e.g., different thicknesses at two or more positions along the length of the reinforcing filament 127).

The reinforcing filament 127 may be formed of a different material than a material of another portion of the endoprosthesis 100 (e.g., a different than a material of the body portion 110 and/or than a material of the polymeric covering 160). For instance, the reinforcing filament 127 may be formed of a different material than at least the body portion 110 of the endoprosthesis 100. Thus, the reinforcing filament 127 may have a different material property (e.g., a higher tensile strength) than a corresponding material property of the body portion 110 of the endoprosthesis 100. For example, the reinforcing filament may be formed of a different material (e.g., a material having a higher tensile strength) than a material of the single wire 120 forming the body portion 110 of the endoprosthesis 100. Thus, the presence of the reinforcing filament 127 may alter the overall properties of the endoprosthesis 100, for example, increasing an overall tensile strength of the endoprosthesis 100, thereby improving the characteristics of the endoprosthesis 100 (e.g., mitigating any foreshortening of the endoprosthesis 100). For example, the presence of the reinforcing filament may prevent any elongation when the endoprosthesis 100 is constrained for delivery, and thereby prevent any subsequent foreshortening of the endoprosthesis 100 when the endoprosthesis 100 is radially expanded at a delivery location. In some embodiments, the reinforcing filament 127 may have a tensile strength of about 500 pounds per square inch or more, about 1,000 pounds per square inch or more, about 1,500 pounds per square inch or more, or about 2,000 pounds per square inch or more, for example. In certain embodiments, the reinforcing filament 127 may possess a tensile strength several orders of magnitude greater than that of the polymeric covering 160. For instance, the reinforcing filament 127 could exhibit a tensile strength at least two, three, four, five, six, seven, eight, nine, or ten times greater than that of the polymeric covering 160. For example, in some embodiments, the tensile strength of the reinforcing filament 127 may be in a range from about 2,000 to about 3,000 pounds per square inch, while that of the polymeric covering 160 may be in a range from about 200 to about 300 pounds per square inch, among other possibilities.

In some embodiments, the reinforcing filament 127 may be manifested as an elongated planar strip, as illustrated in FIGS. 1A-1D. Having the reinforcing filament 127 be manifested as an elongate planar strip can promote aspects herein (e.g., mitigation of any foreshortening experienced by the endoprosthesis 100), and yet can maintain a relatively small or thin profile of the endoprosthesis 100. However, other configurations of the reinforcing filament 127 are possible. For instance, in some embodiments, the reinforcing filament 127 can be manifested as an elongate rod or other structure that can be interwoven with plurality of circumferential segments 130 of the single wire 120.

The presence of the reinforcing filament 127 can promote aspects herein such as promoting mitigation of any foreshortening experienced by the endoprosthesis 100, promoting conformability (e.g., to body lumen) of the endoprosthesis 100, and/or mitigating any forces (e.g., axial and/or radial forces) imparted on the endoprosthesis 100. As illustrated in FIG. 1A, the reinforcing filament 127 can extend substantially longitudinally along the body portion 110. For instance, the reinforcing filament 127 may extend continuously and substantially longitudinally along some or all of a length (in the same direction as the central longitudinal axis 102) of the body portion 110. For example, in some embodiments the reinforcing filament 127 may have a length that is substantially equal to at least half a length of the body portion 110. However, in some embodiments the reinforcing filament 127 may have a length that is substantially equal to a length of the body portion 110, as illustrated in FIG. 1A. Having a length of the reinforcing filament 127 be substantially equal to a length of the body portion 110 can promote aspects herein such as promoting mitigation of any foreshortening experienced by the endoprosthesis 100, promoting conformability of the endoprosthesis 100, and/or mitigating any forces (e.g., axial and/or radial forces) imparted on the endoprosthesis 100 along an entire length of the endoprosthesis 100.

In some embodiments, the reinforcing filament 127 may be interwoven with the single wire 120. For example, the reinforcing filament 127 may be interwoven with two or more plurality of circumferential segments 130 of the single wire 120. For instance, as illustrated in FIG. 1A, and as described in greater detail in FIGS. 1B-1D, the reinforcing filament 127 may be interwoven in an alternating over and under fashion with the plurality of circumferential segments 130 of the single wire 120. For example, as detailed in FIG. 1B the reinforcing filament 127 may be pass along (e.g., be in direct contact with) a given side (e.g., a bottom side) of a first medial circumferential segment 130ca and may pass substantially longitudinally along (e.g., be in direct contact with) a different side (e.g., a top side) of a second medial circumferential segment 130cb that is adjacent (along a central longitudinal axis of the endoprosthesis 100) to the first medial circumferential segment 130ca. The reinforcing filament 127 can continue to pass substantially longitudinally along a side of additional medial circumferential segments 130c in this alternating over and under fashion. For instance, the reinforcing filament 127 can pass along at least each of the medial circumferential segments 130c in an alternating over and under fashion. Having the reinforcing filament 127 pass along at least each of the medial circumferential segments 130c in an alternating over and under fashion can promote aspects herein such as promoting mitigation of any foreshortening experienced by the endoprosthesis 100, promoting conformability of the endoprosthesis 100, and/or mitigating any forces (e.g., axial and/or radial forces) imparted on the endoprosthesis 100 along an entire length of the endoprosthesis 100. In some embodiments, the reinforcing filament 127 may pass along at least each of the medial circumferential segments 130c, the first end circumferential segment 130a, and/or the second end circumferential segment 130b that are located along a common longitudinally extending plane. For instance, in some embodiments, the reinforcing filament 127 may pass along and be interwoven each of the circumferential segments 130 (e.g., each of the medial circumferential segments 130c), the first end circumferential segment 130a, and the second end circumferential segment 130b that are located along a common longitudinally extending plane, as illustrated in FIG. 1A. For example, the reinforcing filament 127 may pass along each of the circumferential segments 130 in an alternating over and under fashion, as illustrated in FIG. 1A. That is, the reinforcing filament 127 may be interwoven in an alternating over and under fashion longitudinally with the plurality of circumferential segments 130 of the single wire 120. However, other configurations are possible. For instance, the reinforcing filament 127 may pass by the same respective side (e.g., top) of two or more consecutive of circumferential segments 130 of the single wire 120 prior to passing by a different side (e.g., bottom) of another of circumferential segments 130 of the single wire 120, for instance as illustrated in FIG. 7. That is the reinforcing filament 127 can pass “over” a first subset of the circumferential segments 130 of the single wire 120 and can pass “under” a second subset (e.g., some or all) of the remaining circumferential segments 130 of the single wire. In any case, interweaving the reinforcing filament 127 with some or all of the plurality of circumferential segments 130 of the single wire 120 can promote aspects herein.

FIG. 1C is a section view (taken along section line 129) of the portion of the endoprosthesis 100 in FIG. 1B, while FIG. 1D is another section view (taken along section line 139) of the portion of the endoprosthesis 100 in FIG. 1B. That is, FIG. 1C illustrates a portion of the medial circumferential segment 130ca, while FIG. 1D illustrates a portion of the medial circumferential segment 130cb. In some embodiments, the reinforcing filament 127 can be in direct contact with filament such as being in direct contact with the plurality of circumferential segments. For example, the reinforcing filament 127 can be in direct contact with at least a plurality of medial circumferential segments (e.g., can be in direct contact with each circumferential segment of the plurality of circumferential segment). As used herein, “direct” contact refers to the absence of the intervening materials. For instance, as illustrated in FIGS. 1C-1D, the reinforcing filament 127 can be in direct contact with a side 162 (bottom side) of the medial circumferential segment 130ca and can be in direct contact with a different respective side 164 (top side) of the medial circumferential segment 130cb.

While FIG. 1A illustrates the presence of an individual reinforcing filament 127, in some embodiments, the endoprosthesis 100 can include a plurality of reinforcing filaments 127. For instance, FIGS. 2-7, illustrate the endoprosthesis 100 including a plurality of reinforcing filaments. As illustrated in FIGS. 2-7, each of the plurality of reinforcing filaments 127 can extend substantially longitudinally along the body portion 110. For instance, each of the plurality of reinforcing filaments 127 may extend coaxially with or parallel to a central longitudinal axis of the endoprosthesis 100. However, in some embodiments a reinforcing filament may extend at an angle or be transverse to the central longitudinal axis. For instance, in some embodiments some or all of the reinforcing filaments may pass helically along the body portion 110, among other possibilities.

As illustrated in FIGS. 2-7, the plurality of reinforcing filaments 127 may be spaced apart circumferentially about the body portion 110. For instance, each of the reinforcing filaments 127 may be spaced apart (e.g., radially) an equal distance from a radially or circumferentially from an adjacent reinforcing filament 127, among other possible configurations. For instance, the plurality of reinforcing filaments 127 may include a first reinforcing filament 127a and a second reinforcing filament 127b, where the first reinforcing filament and the second reinforcing filament may be spaced apart circumferentially about the body portion 110.

In some embodiments, the first reinforcing filament 127a and a second reinforcing filament 127b can be interwoven with corresponding portions of the plurality of circumferential segments in the same manner. For instance, the first reinforcing filament 127a and a second reinforcing filament 127b can each pass “under” or “over” a corresponding portion of the same circumferential segment, as illustrated in FIGS. 2-7. Having the first reinforcing filament 127a and a second reinforcing filament 127b can be interwoven with corresponding portions of the plurality of circumferential segments in the same manner can promote aspects herein. However, in some embodiments, the reinforcing filament 127a and a second reinforcing filament 127b can be interwoven with corresponding portions of the plurality of circumferential segments in the different manner. For instance, a first reinforcing filament 127a can pass “over” a respective portion of a circumferential segment and the second reinforcing filament 127b can pass “under” a respective portion of the same circumferential segment.

In some embodiments, the plurality of reinforcing filaments may be the same shape, the same size, or both the same shape and the same size. Having the plurality of reinforcing filaments be the same size, same shape, or both, can promote aspects herein. For instance, in some embodiments, each of the plurality of reinforcing filaments may be the same shape and same size (e.g., may have the same length along the central longitudinal axis 102 of the endoprosthesis 100, same width along a direction that is traverse to the central longitudinal axis 102 of the endoprosthesis 100, and may have the same thickness). However, in some embodiments one or more of the plurality of reinforcing filaments may have a different shape and/or size than another one of the plurality of reinforcing filaments. In some embodiments, each of the plurality of reinforcing filaments may be interwoven with the plurality of circumferential segments, as illustrated in FIGS. 2-7.

In some embodiments, the endoprosthesis 100 may comprise a polymeric covering 160, shown in the figures via dotted shading, coupled to the body portion 110, the single wire 120, and/or the plurality of circumferential segments 130. In some embodiments, the polymeric covering 160 may be fixedly attached to the body portion 110, the single wire 120, and/or the plurality of circumferential segments 130.

In some embodiments, the polymeric covering 160 may extend along an inner surface of the endoprosthesis 100, the body portion 110, the single wire 120, the reinforcing filament 127, and/or the plurality of circumferential segments 130. In some embodiments, the polymeric covering 160 may extend along an outer surface of the endoprosthesis 100, the body portion 110, the single wire 120, the reinforcing filament 127 (e.g., each of a plurality of reinforcing filament 127), and/or the plurality of circumferential segments 130. In at least some embodiments, the body portion 110, the single wire 120, the reinforcing filament, and/or the plurality of circumferential segments 130 may be embedded within the polymeric covering 160. For instance, as illustrated in FIGS. 1A-1D, each of the body portion 110, the single wire 120, the reinforcing filament, and the plurality of circumferential segments 130 (e.g., the medial circumferential segments 130ca and 130cb) may be embedded within the polymeric covering. In some embodiments, the anti-migration element (not illustrated) and/or a plurality of anti-migration elements (not illustrated), etc. may be embedded within the polymeric covering 160. Other configurations, including combinations thereof, are also contemplated. Some suitable but non-limiting examples of polymeric materials for the polymeric covering 160 are discussed below.

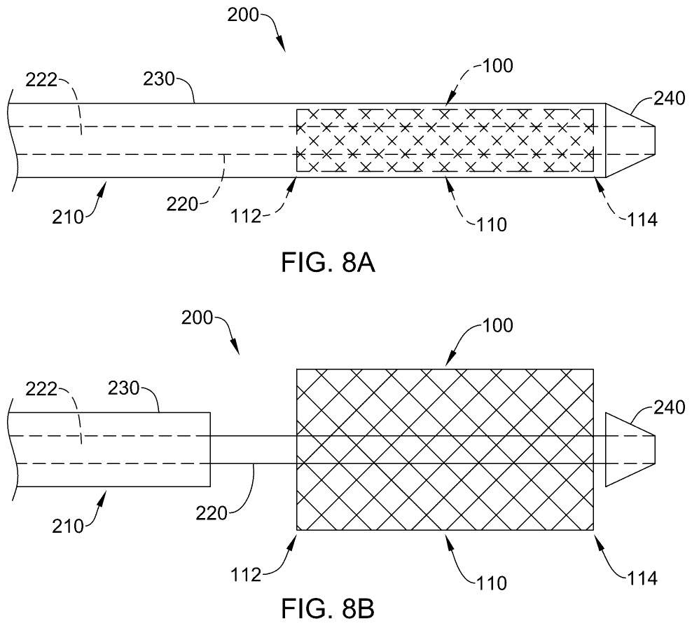

FIGS. 8A-8B illustrate selected aspects of an endoprosthesis system 200 comprising the endoprosthesis 100 and a delivery device 210. The endoprosthesis 100 can be analogous to or similar to the endoprosthesis 100 described with respect to FIGS. 1A-1B and/or FIGS. 2-7, herein.

In some embodiments, the delivery device 210 may comprise an inner tubular member 220 and an outer tubular member 230. The endoprosthesis 100 may be disposable within an annular space disposed between the inner tubular member 220 and the outer tubular member 230 (e.g., within a lumen of the outer tubular member 230 and around and/or radially outward of the inner tubular member 220) in a collapsed delivery configuration. The inner tubular member 220 and the outer tubular member 230 may be axially movable relative to each other to load and/or deploy the endoprosthesis 100. In some embodiments, the inner tubular member 220 may comprise a guidewire lumen 222 extending therein. In some embodiments, the delivery device 210 may comprise a distal tip 240 disposed at a distalmost end of the inner tubular member 220. In at least some embodiments, the guidewire lumen 222 may extend through the distal tip 240. In alternative some embodiments, the delivery device 210 may comprise a crochet delivery device. Other configurations are also contemplated.

In some embodiments, the endoprosthesis 100 may be configured to shift between the collapsed delivery configuration, as seen in FIG. 8A, and an expanded deployed configuration, as seen in FIG. 8B. In some embodiments, the endoprosthesis 100 may be configured to self-expand from the collapsed delivery configuration to the expanded deployed configuration. That is, the implantable medical devices herein may be a self-expanding device such as known or heretofore known to those of ordinary skill in the art. For instance, the implantable medical device may be formed of shape-memory or heat-formable material (e.g., Nitinol or Elgiloy® or shape memory polymers) so that the implantable medical devices herein return to a pre-shaped expanded configuration from a collapsed configuration upon advancement from a delivery sheath (any acceptable tubular elongated member such as known to those of ordinary skill in the art for delivery of medical devices) and/or withdrawal of a delivery sheath which maintains the implantable medical devices in a delivery configuration therein. In some embodiments, the endoprosthesis 100 may be balloon expandable from the collapsed delivery configuration to the expanded deployed configuration. Other configurations are also contemplated.

In some embodiments, the body portion 110 and/or the endoprosthesis 100 may be configured to minimize and/or avoid foreshortening and/or a change in the length of the body portion 110 and/or the endoprosthesis 100 as the body portion 110 and/or the endoprosthesis 100 shifts between the collapsed delivery configuration and the expanded deployed configuration. In some embodiments, the length of the body portion 110 and/or the endoprosthesis 100 from the first end 112 to the second end 114 may be configured to change by less than 10% when shifting between the collapsed delivery configuration and the expanded deployed configuration. In some embodiments, the length of the body portion 110 and/or the endoprosthesis 100 from the first end 112 to the second end 114 may be configured to change by less than 7.5% when shifting between the collapsed delivery configuration and the expanded deployed configuration. In some embodiments, the length of the body portion 110 and/or the endoprosthesis 100 from the first end 112 to the second end 114 may be configured to change by less than 5% when shifting between the collapsed delivery configuration and the expanded deployed configuration. In some embodiments, the length of the body portion 110 and/or the endoprosthesis 100 from the first end 112 to the second end 114 may be configured to change by less than 2.5% when shifting between the collapsed delivery configuration and the expanded deployed configuration. In some embodiments, the length of the body portion 110 and/or the endoprosthesis 100 from the first end 112 to the second end 114 may be configured to change by less than 1% when shifting between the collapsed delivery configuration and the expanded deployed configuration. Other configurations are also contemplated.

The endoprosthesis 100 may be configured to bend into a U-shape and/or an S-shape without kinking. This may provide an advantageous benefit in deployment of the endoprosthesis 100 as endoprosthesis 100 is able to comply with the tortuous anatomy of a patient or subject as the endoprosthesis 100 is guided toward its terminal destination of deployment within a patient or subject. In other words, when endoprosthesis 100 is deployed, for instance through a vessel or other body lumen, the endoprosthesis 100 is able to bend along the curvature of the body lumen as the endoprosthesis 100 is positioned through the body lumen of a patient or subject without causing undue trauma to the tissue of the patient or subject (e.g., lumens, vessels, organs, organ tissue, lumen walls, vessel walls, etc.). Owing to the weave of the filament and flexibility of covering (e.g., polymeric covering), the endoprosthesis 100 is also able to revert into its radially expanded shape upon deployment, and maintain patency of a lumen, vessel, duct or the like at its terminal destination of deployment. In other words, the endoprosthesis 100 may be delivered in a straight configuration for delivery into a patient through a device such as a catheter. As the endoprosthesis 100 is navigated through the body lumen of a patient or subject, the endoprosthesis 100 is able to bend with the curves of the body lumen of the patient or subject without causing undue trauma to the body lumen of the patient or subject. Upon reaching the desired area of treatment, the endoprosthesis 100 may revert to its original radially expanded shape or configuration, or may further conform to the geometry of the desired area of treatment. For instance, and by non-limiting example, the endoprosthesis 100 may conform to a bend of the bile duct and/or pancreatic duct while maintaining patency in the bile duct or pancreatic duct.

Embodiments herein provide method of forming an endoprosthesis (e.g., method of forming the endoprosthesis 100). The method can include receiving a body portion such as the body portion 110, described herein. For instance, the body portion may extend axially from a first end to a second end along a central longitudinal axis; wherein the body portion is formed from a single wire extending from the first end to the second end; and the single wire forms a plurality of circumferential segments extending non-helically around the central longitudinal axis, as described herein. In some embodiments, the body portion can be formed on a mandrel, among other possibilities.

The method can include interweaving a reinforcing filament with the plurality of circumferential segments of the single wire to form a reinforced framework. For instance, the method can include interweaving the reinforcing filament in an alternating over and under fashion with the plurality of circumferential segments, as described herein. In some instances, the method can include interweaving an individual reinforcing filament (e.g., only one reinforcing filament) with the plurality of circumferential segments of the single wire to form a reinforced framework including an individual reinforcing filament. However, in some instances the method can include interweaving a plurality reinforcing filaments with the circumferential segments of the single wire to form a reinforced framework including the plurality of reinforcing filaments.

Subsequent to formation of the reinforced framework, the method can include covering the reinforced framework with a polymeric covering to form the stent. In some embodiments, covering the reinforced framework with a polymeric covering may include overlaying the reinforced framework with at least one solid circumferential polymeric sleeve; and heating the solid circumferential polymeric sleeve to cause the solid circumferential polymeric sleeve to reflow and form the polymeric covering. For example, the method can include covering the reinforced framework with a solid polymeric sleeve that overlays all of the reinforcing filament in the reinforced framework. Heating the at least one solid circumferential polymeric covering to a given temperature (e.g., 100 degrees Celsius, etc.) may cause the circumferential sleeve to reflow and thereby form a polymeric covering overlaying at least a portion of the reinforced framework (e.g., such that at least a portion of the reinforced framework is embedded in the polymeric covering). In some embodiments, the polymeric covering (e.g., formed from a circumferential sleeve) can be formed of polyurethane, silicone, or a combination thereof, among other possible materials. For instance, the circumferential polymeric sleeve can be formed of polyurethane. However, in some embodiments the circumferential polymeric sleeve can be formed of silicone.

In some embodiments, the circumferential polymeric sleeve can be manifested as an individual sleeve (e.g., disposed circumferentially about an exterior surface or being disposed about an interior (e.g., intraluminal surface) of the reinforced framework. However, in some embodiments, the circumferential polymeric sleeve can be manifested as two or more sleeves, such as a first sleeve (e.g., a polyurethane sleeve) disposed on an inside surface (e.g., intralumenal surface) of the reinforced framework and a second sleeve (e.g., a silicone sleeve) disposed about an exterior surface of the reinforced framework, among other possibilities.

However, other methods of forming the polymeric covering are possible. For instance, in some embodiments the polymeric covering can for formed by applying a liquid polymeric covering to the reinforced framework. Applying the liquid polymeric covering may take the form of dip coating, spray coating, or otherwise applying a liquid polymeric covering to the reinforced framework such that the liquid polymeric covering subsequently becomes the polymeric covering on the reinforced framework. In any case, at least due to the presence of the reinforced framework (e.g., an interwoven reinforcing filament therein) in conjunction with the other elements of the endoprosthesis, a length of the body portion from the first end to the second end is configured to change by less than 5% when shifting between a collapsed delivery configuration and an expanded deployed configuration, as described herein.

The materials that can be used for the various components of the endoprosthesis and/or the endoprosthesis system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the delivery device, the endoprosthesis, the single wire, the polymeric covering, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An endoprosthesis, comprising:
a body portion extending axially from a first end to a second end along a central longitudinal axis; wherein the body portion is formed from a single wire extending from the first end to the second end; and wherein the single wire forms a plurality of circumferential segments extending non-helically around the central longitudinal axis; and
a reinforcing filament extending substantially longitudinally along the body portion.

2. The endoprosthesis of claim 1, wherein the reinforcing filament has a length that is substantially equal to a length of the body portion.

3. The endoprosthesis of claim 1, wherein the reinforcing filament is interwoven with the plurality of circumferential segments of the single wire.

4. The endoprosthesis of claim 3, wherein the reinforcing filament is interwoven in an alternating over and under fashion with the plurality of circumferential segments of the single wire.

5. The endoprosthesis of claim 3, wherein the reinforcing filament is in direct contact with the plurality of circumferential segments.

6. The endoprosthesis of claim 3, wherein the plurality of circumferential segments includes a plurality of medial circumferential segments, and wherein the reinforcing filament is interwoven with at least each of the plurality of medial circumferential segments.

7. The endoprosthesis of claim 1, wherein the reinforcing filament is an elongated planar strip.

8. The endoprosthesis of claim 1, wherein the reinforcing filament is formed of polyester, polytetrafluoroethylene, or a combination thereof.

9. The endoprosthesis of claim 1, wherein the reinforcing filament is included in a plurality of reinforcing filaments, and wherein each reinforcing filament of the plurality of reinforcing filaments extends substantially longitudinally along the body portion.

10. The endoprosthesis of claim 9, wherein respective reinforcing filaments of the plurality of reinforcing filaments are spaced apart circumferentially about the body portion.

11. The endoprosthesis of claim 9, wherein the plurality of reinforcing filaments comprises: a first reinforcing filament and a second reinforcing filament, wherein the first reinforcing filament and the second reinforcing filament are spaced apart circumferentially about the body portion.

12. The endoprosthesis of claim 9, wherein respective reinforcing filaments of the plurality of reinforcing filaments are the same shape.

13. The endoprosthesis of claim 9, wherein respective reinforcing filaments of the plurality of reinforcing filaments are the same size.

14. The endoprosthesis of claim 1, further comprising a polymeric covering fixedly attached to the body portion, wherein the reinforcing filament is embedded in the polymeric covering.

15. The endoprosthesis of claim 1, wherein a length of the body portion from the first end to the second end is configured to change by less than 5% when shifting between a collapsed delivery configuration and an expanded deployed configuration.

16. A self-expanding endoprosthesis, comprising:
a body portion extending axially from a first end to a second end along a central longitudinal axis; wherein the body portion is formed from a single wire extending from the first end to the second end; and wherein the single wire forms a plurality of circumferential segments extending non-helically around the central longitudinal axis;
a plurality of reinforcing filaments extending substantially longitudinally along the body portion, wherein respective reinforcing filaments of the plurality of reinforcing filaments are interwoven with the plurality of circumferential segments and are formed of a different material than the body portion; and
a polymeric covering fixedly attached to the body portion, wherein the plurality of reinforcing filaments and the body portion are embedded in the polymeric covering.

17. The self-expanding endoprosthesis of claim 16, wherein:
each circumferential segment of the plurality of circumferential segments comprises an undulating arrangement of first struts and second struts defining peaks and valleys, the first struts being disposed at a first angle relative to the central longitudinal axis in a side view of the body portion and the second struts being disposed at a second angle relative to the central longitudinal axis in the side view of the body portion;
each circumferential segment of the plurality of circumferential segments is connected to an axially adjacent circumferential segment of the plurality of circumferential segments by a transition segment of the single wire disposed at a third angle relative to the central longitudinal axis in the side view of the body portion, wherein the third angle is different from the first angle and the second angle; and
all transition segments of the single wire collectively define a transition zone.

18. The self-expanding endoprosthesis of claim 17, wherein the transition zone extends:
parallel to the central longitudinal axis; and
helically around the central longitudinal axis.

19. A method of forming an endoprosthesis, the method comprising:
receiving a body portion extending axially from a first end to a second end along a central longitudinal axis; wherein the body portion is formed from a single wire extending from the first end to the second end; and wherein the single wire forms a plurality of circumferential segments extending non-helically around the central longitudinal axis;
interweaving a reinforcing filament with the plurality of circumferential segments of the single wire to form a reinforced framework, wherein the reinforcing filament is formed of a different material than the body portion; and
covering the reinforced framework with a polymeric covering to form the endoprosthesis, wherein a length of the body portion from the first end to the second end is configured to change by less than 5% when shifting between a collapsed delivery configuration and an expanded deployed configuration.

20. The method of claim 19, wherein covering the reinforced framework with the polymeric covering further comprises applying a liquid polymeric covering to the reinforced framework.
